(19) United States
(12) Patent Application Publication
(10) Pub. No.: US 2016/0361333 A1
(43) Pub. Date: Dec. 15, 2016

(54) USE OF CITRUS POLYPHENOL FOR WOUND HEALING AND COMPOSITION THEREOF

(71) Applicant: National Taiwan University, Taipei (TW)

(72) Inventors: Hsiao-Cheng Tsai, Taipei (TW); Yi-Chen Li, Taipei (TW); Sheng-Hao Hsu, Taipei (TW); Tai-Horng Young, Taipei (TW); Min-Huey Chen, Taipei (TW)

(73) Assignee: NATIONAL TAIWAN UNIVERSITY, Taipei (TW)

(21) Appl. No.: 14/874,052

(22) Filed: Oct. 2, 2015

(30) Foreign Application Priority Data
Jun. 9, 2015 (TW) 104118574

(51) Int. Cl. A61K 31/7016; A61K 31/353; A61K 36/752

(52) U.S. Cl. CPC A61K 31/7016; A61K 36/752; A61K 31/353

(57) ABSTRACT

The present invention provides a composition for wound healing, which includes an effective amount of a citrus polyphenol. Moreover, the present invention also provides a method for improving wound healing and improving fibroblasts migration and/or proliferation, which includes applying an effective amount of citrus polyphenol to the wound and fibroblasts.

### CROSS-REFERENCE TO RELATED APPLICATION

This application claims foreign priority under 35 U.S.C. §119(a) to patent application Ser. No. 104118574, filed on Jun. 9, 2015, in the Intellectual Property Office of Ministry of Economic Affairs, Republic of China (Taiwan, R.O.C.), the entire content of the above-referenced application is incorporated herein by reference.

### BACKGROUND OF THE INVENTION

1. Field of the Invention

The present invention relates to uses of a citrus polyphenol and, especially, to a use of a citrus polyphenol for preparing a composition for wound healing

2. Description of Related Art

Wound healing is a complicated dynamic process, and the ideal healing situation would be to restore the structure, function and appearance of the normal tissues. In general, with proper treatment, it will take one to two weeks for a wound to heal. If the wound is not properly treated, it would slow down the wound healing process and lead to the occurrence of ulcer. If it is not a severe wound, the improper healing of the wound may lead to permanent scar. If it is a severe wound, the improper healing of the wound may lead to the treatments of wound debridement surgery, grafting, or amputation to preserve the life of the patient due to the occurrence of cellulitis.

Mouth ulcer is a common oral health problem in adult among various wounds. Ulcer is a symptom of various oral diseases and leads to patients' severe pain and un-comfort. The general causes of mouth ulcer are physical injuries, vitamin deficiency, bacterial infection, and so on. General speaking, the healing of an ulcer spanning a small area may take less than a week. It may take several months for the healing of an ulcer spanning an area with a diameter of greater than 1 cm.

The process and required time of healing of wound or ulcers are impacted by several factors, such as the type and size of the wound, the nutrition condition and age of the patient, other system diseases, and drug usages. Most of the conventional technologies for treating ulcers are relevant to supportive play chotherapy, local treatment of steroid, or medicine for killing bacteria. They are useful for reducing the area of ulcer, avoiding repetitive infection, and reducing the time required for healing.

However, anti-inflammation steroid drug is not suitable for patients with bacteria/virus infected ulcer and patients with immunity-deficiency diseases. The medicaments with the ability of killing bacteria, such as alcohol-containing liquid medicaments for external uses or povidone-iodine, may destroy and damage fibroblasts which are required for wound healing.

Fibroblasts play crucial roles during wound healing. When the existence of a wound is detected, fibroblasts will be activated to become polygonal phenotype as discussed by Li Y C et al. in “Pearl extract enhances the migratory ability of fibroblasts in a wound healing model” (Pharm. Biol., 2013, 51:289-297). When the wound is healing, fibroblasts will proliferate and move to the location of the wound for repairing as discussed by Khovidhunkit, S. O. et al. in “In vitro study of the effects of plaunotol on oral cell proliferation and wound healing” (J. Asian Nat. Prod. Res., 2011, 13:149-159). The increased quantity of fibroblasts improved the condition of wound healing in experimental models as discussed by Lamme E. N. et al. in “Higher numbers of autologous fibroblasts in an artificial dermal substitute improve tissue regeneration and modulate scar tissue formation” (J. Pathol., 2000, 1900:595-603).

It is known that several polyphenol compounds extracted from plants have various physiological effects. For example, Ferruelo et al. discussed the abilities of resveratrol and other wine polyphenols in inhibiting the proliferation of prostate adenocarcinoma cells (LNCaP) in “Effects of resveratrol and other wine polyphenols on the proliferation, apoptosis and androgen receptor expression in LNCaP cells” (Actas Urológicas Españolas (English Edition) 2014, 38:397-404). Schoene et al. discussed the effects of polymeric polyphenols extracted from cinnamon in inhibiting the proliferation of hematologic tumor cell lines in “Water-soluble polymeric polyphenols from cinnamon inhibit proliferation and alter cell cycle distribution patterns of hematologic tumor cell lines” (Cancer Letters, 2005, 230:134-140). Leifert et al. discussed the abilities of grape seed and red wine polyphenol extracts in inhibiting the cell proliferation in “Grape seed and red wine polyphenol extracts inhibit cellular cholesterol uptake, cell proliferation, and 5-lipoxygenase activity” (Nutrition Research, 2008, 28:842-850).

Chen et al. further indicated the abilities of epigallocatechin-3-gallate, luteolin, apigenin, myricetin, quercetin, and cyanidin in inhibiting retinal pigment epithelial cells in “Effects of the vegetable polyphenols epigallocatechin-3-gallate, luteolin, apigenin, myricetin, quercetin, and cyanidin in primary cultures of human retinal pigment epithelial cells” (Molecular Vision, 2014, 20:242-258). More studies indicated the abilities of epigallocatechin-3-gallate in inhibiting the proliferation of aortic smooth cells: (Z. Shu, M. Yu, G. Zeng, X. Zhang, L. Wu, X. Tan, 2014, “Epigallocatechin-3-gallate inhibits proliferation of human aortic smooth muscle cells via up-regulating expression of mitofusin,” European Journal of Cell Biology, 93:137-144; P. L. Liu, J. T. Liu, H. F. Kuo, I. W. Chong, and C. C. Hsieh, “Epigallocatechin Gallate Attenuates Proliferation and Oxidative Stress in Human Vascular Smooth Muscles Cells Induced by Interleukin-1β via Heme Oxygenase-1,” 2014, Mediators of Inflammation, Article ID 523684, http://dx.doi.org/10.1155/2014/523684).

However, the common additives of the conventional oral composition (such as mouthwash) are tea and bamboo polyphenols with the function of killing bacteria in dental plaque and preventing tooth cavity. These additives do not include the plant polyphenol compounds which can improve the proliferation of fibroblasts as disclosed in prior arts. In current treatment methods for the healing of wounds or ulcers, chemicals with side effects are used mainly. They tend to lead to the increased risks of patient's discomfort or delaying the time required for healing. It is desired to find a method to effectively improve the wound healing without other undesired results.

### SUMMARY OF THE INVENTION

Due to the aforementioned problems to be resolved, the present invention provides a use of citrus polyphenol for preparing a composition to improve wound healing, wherein the composition includes an effective amount of citrus polyphenol and pharmaceutically acceptable carriers.

According to the embodiments of the present invention, the citrus polyphenols include delphinidin, pelargonidin, peonidin, malvidin, cyaniding, narirutin, naringin, hesperidin, and neohesperidin.

According to the embodiments of the present invention, the wounds include skin injuries, burns, skin ulcer, or oral ulcer. Based on one embodiment of the present invention, the composition can be used to improve the proliferation and/or migration of the fibroblasts to further improve the healing of wounds.

According to the embodiment of the present invention, the content of citrus polyphenols is from greater than 0 wt % to less than 1 wt % based on the total weight of the composition. In one embodiment, the content of citrus polyphenols is from greater than 0 wt % to less than or equal to 0.1 wt %. In another embodiment, the content of citrus polyphenols is 0.01 wt %.

In addition, in another aspect of the present invention, the present invention provides a use of a citrus polyphenol for preparing a composition to improve the proliferation and/or migration of fibroblasts.

According to the embodiments of the present invention, the composition is used as an oral composition. According to another embodiment of the present invention, the composition is in a dosage form selected from the group consisting of mouthwash, tooth powder, tooth paste, dental gel, periodontal gel, chewable tablet, thin film, oral strip, oral gel, oral tablet, and foaming tablet.

According to the embodiments of the present invention, the pharmaceutically acceptable carrier is at least one selected from the group consisting of foaming agent, disintegration agent, excipient, viscosity regulator, diluent, surfactant, pH modifier, grinding agent, wetting agent, mouthfeel agent, sweetener, spice, coloring agent, preservative, stabilizer, and anti-bacteria agent.

### BRIEF DESCRIPTION OF THE DRAWINGS

FIG. 1A is images of Hs68 cells after the treatment of different concentrations of citrus polyphenols for 4 hours under optical microscope. FIG. 1B is images of Hs68 cells after the treatment of different concentrations of citrus polyphenols for 3 days under optical microscope. FIG. 1C is images of Hs68 cells after the treatment of different concentrations of citrus polyphenols for 6 days under optical microscope. The scale of the optical microscope image is 100 μm.

FIG. 2A showed the test results of MTT for Hs68 cells in media with different concentrations of citrus polyphenols. FIG. 2B showed the test results of lactic dehydrogenase (LDH) assay for Hs68 cells in media with different concentrations of citrus polyphenols, *p<0.01 indicated the results with significant differences.

FIG. 3A indicated the dynamic process of Hs68 cells under scratch test for 16 hours in control media without citrus polyphenols. FIG. 3B indicated the dynamic process of Hs68 cells under scratch test for 16 hours in media containing 0.1% citrus polyphenols. FIG. 3C indicated the dynamic process of Hs68 cells under scratch test for 16 hours in media containing 0.01% citrus polyphenols. FIG. 3D showed the average quantity of Hs68 cells which relocated to the location of the scratches after 16 hours during the scratch test in media containing different concentrations of citrus polyphenols. The scale of FIG. 3A-3C is 100 g m, **p<0.01 indicated the results with significant differences.

FIGS. 4A, 4B and 4C are images of Hs68 cells under optical microscope during scratch tests in media containing 0%, 0.1% or 0.01% citrus polyphenols respectively for 3 days. The scale of the optical microscope image is 100 μm.

### DETAILED DESCRIPTION OF THE PREFERRED EMBODIMENTS

The following specific examples are used for illustrating the present invention. A person skilled in the art can easily conceive the other advantages and effects of the present invention based on the disclosure of this specification. The present invention can be implemented through different specific format or application conditions, and such instruction details can be modified or adapting different view-points and applications without departing from the spirit of the present invention.

To be noted, as used in the specification, unless clearly define an indicated object, otherwise the singular formats of “a” and “the” include plural indicated objects. Unless indicated in the context of the specification, otherwise the terminology of “or” is interchangeable with the terminology of “and/or.”

The present invention provides a use of a citrus polyphenol for preparing a composition to improve wound healing. According to the embodiments of the present invention, the wounds include skin injury, burns, skin ulcer, or oral ulcer.

The terminology of “wound” used in the specification indicates the damages of body tissue structures or organs due to chemical or physical external forces. More specifically, the body tissue structures or organs further indicate the mucous tissues of skin or mouth. General speaking, depending on the time required for healing, the wounds can be categorized as acute wounds or chronic wounds. The chronic wound is defined as any wound which does not heal within predicted time period or stays in a specific healing process for a long period of time, such as more than 4 to 6 weeks. The terminology of “oral ulcer” indicates the ulcer site which is derived from the wounds that is not healed properly in time due to the wounds of damaged inner wall of the oral mucous or damaged surface of the tongue.

The citrus polyphenols in the composition used in the present invention contain a mixture of polyphenols which can be extracted from citrus fruits mainly using the extraction method as known in the technical field. According to the embodiments of the present invention, citrus polyphenols contain one or more active substances. These active substances include, not limited to: delphinidin, pelargonidin, peonidin, malvidin, cyaniding, narirutin, naringin, hesperidin, and neohesperidin. Preferably, the citrus polyphenols used in the present invention are a mixture of delphinidin, pelargonidin, peonidin, malvidin, cyaniding, narirutin, naringin, hesperidin, and neohesperidin.

According to the embodiments of the present invention, the content of the citrus polyphenols is from greater than 0 wt % to less than 1 wt % based on the total weight of the composition. In another embodiment, the content of the citrus polyphenols is from greater than 0 wt % to less than or equal to 0.1 wt % based on the total weight of the composition. In another embodiment, preferably, the content of the citrus polyphenols is 0.01 wt % based on the total weight of the composition.

In addition, according to the embodiments of the present invention, besides containing effective amount of the citrus polyphenols, the composition further contains one or more pharmaceutically acceptable carrier. The terminology of “pharmaceutically acceptable carriers” indicates the carriers which are commonly used in medicine in preparing medical composition. The examples of pharmaceutically acceptable carriers include, not limited to: foaming agent, disintegration agent, excipient, viscosity regulator, diluent, surfactant, pH modifier, grinding agent, wetting agent, mouthfeel agent, sweetener, spice, coloring agent, preservative, stabilizer, anti-bacteria agent, and the like.

According to the embodiments of the present invention, the composition can be in the dosage form of liquid, semi-solid, solid or spray. Preferably, the composition can be in the dosage form of powder, granule, liquid, emulsion, cream, ointment, gel, patch, spray, micro-emulsion, or the like.

The appropriate excipients and other materials which can be used in the present invention are commonly known in the technical field and are determined by the desired dosage form of the composition or by the desired tissues for the application of the composition. General speaking, the typical excipients include, not limited to: the substance for the formation of liquid dosage form, such as, water, acetone, ethanol, glycol, propylene glycol, butyl-1,3-diol, isopropyl myristate, isopropyl palmitate, mineral oil or the mixture thereof; the macromolecule thin film (such as polycaprolactone thin film) or macromolecule bulk materials (such as foam sponge) for the formation of solid dosage forms, such as patches or thin films; the macromolecule water-gel (such as chitin water-gel, collagen water-gel, and hyaluronic acid water-gel), the macromolecule micro-particle or liposome, pyrrolidones or the mixture thereof for the formation of solid or gel dosage forms.

According to another embodiment of the present invention, the composition is used in an oral composition. According to an embodiment of the present invention, the oral composition is the formulation of mouthwash, tooth powder, tooth paste, dental gel, periodontal gel, chewable tablet, thin film, oral strip, oral gel, oral tablet, foaming tablet, and the like. For example, when the composition of the present invention is used in mouthwash formulation, the composition can further include the commonly used substances in mouthwash which can be taken orally, such as anti-bacteria agent, food coloring and edible menthol. According to the embodiments of the present invention, the anti-bacteria agents include, not limited to: pharmaceutically acceptable disinfectants of guanidine and quaternary ammonium salt, such as chlorhexidine; compounds of isothiazolin-ketones; or chlorine-containing organic compounds, such as triclosan. In another embodiment, the composition can additionally contain active substances which are not polyphenols, such active substances including, not limited to: amino acids, proteins, peptide, nucleic acids, nutrition formulations, steroid, analgesic agent, anti-inflammatory agents, anti-virus agent, hemostat, anti-allergy agent, or the like.

Depending on the tissues for treatments, the pharmaceutical composition of the present invention can contain additional ingredients, such as: enhancing the delivery of active ingredients to tissues through permeation. The appropriate permeation-enhancing agents include, not limited to: acetone; various alcohols, for example—ethanol, propylene glycol, tetrahydrofuryl alcohol; alkyl sulfoxides, for example—dimethyl sulfoxide; dimethylacetamide; dimethylformamide; polyethylene glycol; pyrrolidones, for example—polyvinylpyrrolidone; urea; and various water-soluble or water-insoluble sugar esters, for example, Tween 80 and so on.

From a different aspect, the present invention provides a method to promote wound healing of the tested subject, including the application of the composition containing an effective amount of citrus polyphenol to the tested subject. According to the embodiments of the present invention, the composition of the present invention is applied to the wound. After the treatment, the fibroblast cells around the wound will migrate and/or proliferate. These fibroblast cells will gradually migrate and/or proliferate from the surround of the wound to the center of the wound, which promotes wound healing.

The following are specific embodiments further demonstrating the efficacy of the current invention, but not to limit the scope of the current invention.

### EXAMPLES

### Example 1

### Culture Fibroblast Cells with Citrus Polyphenols

Human fibroblast cells, Hs68 (BCRC no. 60038), were cultured according to the methods described by Lou P J et al. (Biomaterials, 2010; 31:1568-1577) and Chung Y C et al. (Biomaterials 2011, 32:4471-4480). Under the condition of 95% air/5% CO2 at 37° C., Hs68 cells were cultured in DMEM medium with 10% fetal bovine serum (FBS) (Biological Industries, Israel) for 3-5 days until the cells reached confluence. Cells were washed with PBS buffer, and treated with 0.05% trypsin for 5 mins at 37° C. to dissociate cells. Then spin down the dissociated cells. These cells were subsequently re-suspended with culture medium and plated at the density of approximately 10,000 cell/cm² in 24-well plates (TCPS, Coring). After four hours, when the cells were adhered to the wells, the culture medium was changed to the DMEM medium containing 10% FBS and with 0%, 0.01%, 0.1% or 1% citrus polyphenols (purchased from Fytexia, France), respectively. Subsequently, fresh medium was provided daily.

### Example 2

### The Effect of Citrus Polyphenols on Cellular Morphology of Fibroblast Cells

Observe cellular morphology of Hs68 cells after cultured in citrus polyphenol containing medium using Lieca DM1600 handstand phase contrast microscope.

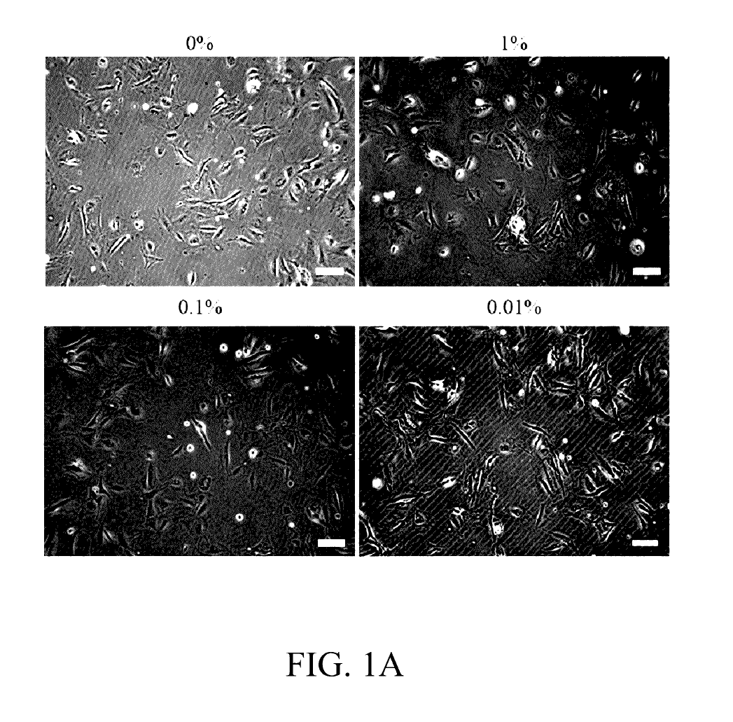

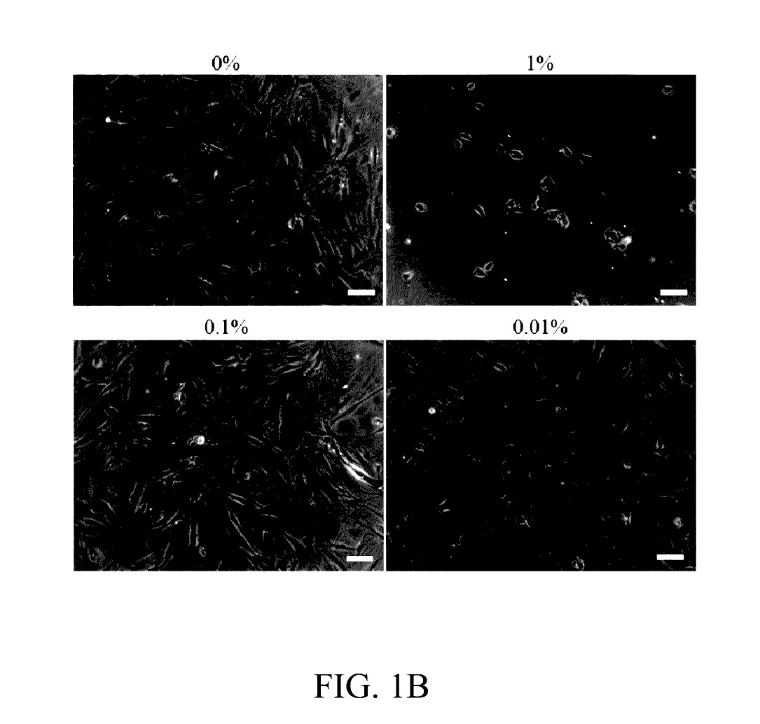

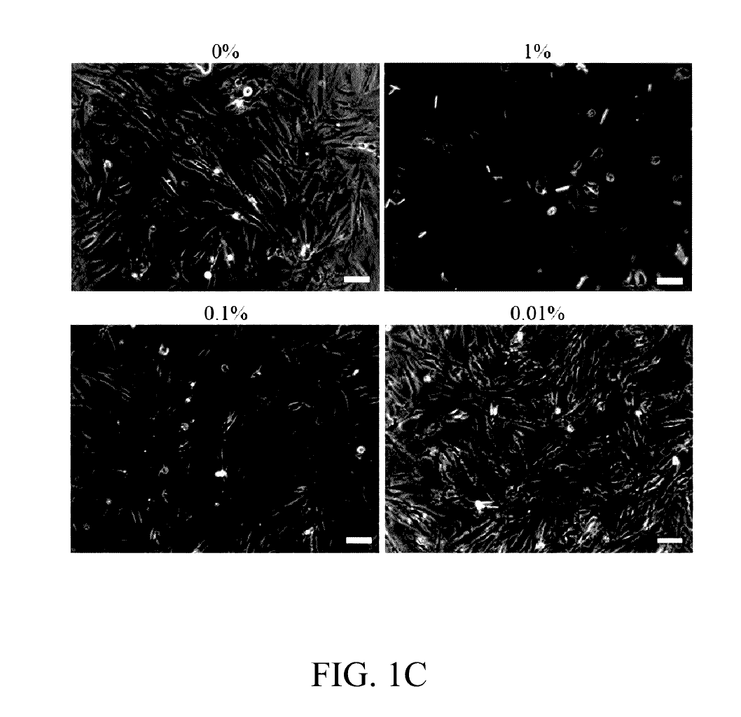

Please refer to FIG. 1A-1C, FIG. 1A-1C show cellular morphology of Hs68 cells treated with different concentrations of citrus polyphenol for 4 hours, 3 days and 6 days, respectively. As shown in FIG. 1B, after 3-day treatment with 0.01 wt % or 0.1 wt % citrus polyphenols, it can be observed that Hs68 cells show proliferation phenotype, and the shape of the cells displays flat polygonal morphology, indicating that the numbers of Hs68 cells increase after treatment of 0.01 wt % or 0.1 wt % citrus polyphenols. In contrast, no proliferation was observed for Hs68 cells treated with 1 wt % citrus polyphenol for the duration of the experiment. Furthermore, as shown in FIG. 1C, Hs68 cells gradually reached confluence after treatment of 0.01 wt % and 0.1 wt % citrus polyphenols for 6 days.

In addition, from FIG. 1A-1C, it can be found that most of Hs68 cells show polygonal shape (active form) and then transform into elongated fibroblast phenotype (inactive form) after gradually reaching confluence under the treatment of 0.01 wt % citrus polyphenols.

### Example 3

### The Effect of Citrus Polyphenols on the Activity of Fibroblast Cells

The survival of Hs68 cells is evaluated by the ability of cells to reduce 3-(4,5-Dimethylthiazol-2-yl)-2,5-diphenyltetrazolium bromide (MTT, Sigma). The mitochondria dehydrogenase in live cells can selectively cleave tetrazole ring to produce the blue/purple formazan crystals. The level of reducing MTT to formazan can reflect the metabolism function of the cells.

In order to perform the MTT test, the original DMEM medium was removed prior to the measurement, and the cells were cultured in 0.2 ml of MTT (5 mg/ml, dissolve in PBS) at 37° C. for 3 hours. After the culturing, the culture medium was taken out and treated with dimethyl sulfoxide (dissolve in PBS), then the mixture was shaking for 15 minutes to dissolve the reaction product, formazan. The formazan solution was detected at 570 nm optical density using ELISA plate reader (M2′, Molecular Devices).

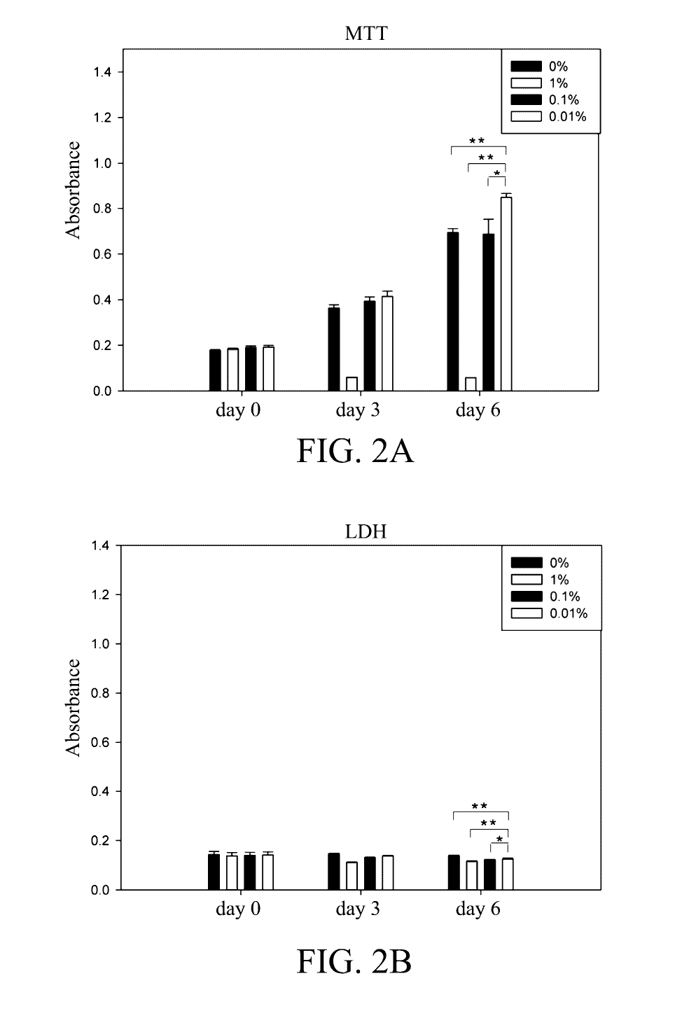

The test results of MTT were shown in FIG. 2A. After 6 days of culturing, the MTT reducing activity of Hs68 cells in medium containing 0.01 wt % citrus polyphenols is significantly higher than those in medium without citrus polyphenols. The results indicated that 0.01 wt % citrus polyphenols is most suitable for the proliferation of Hs68 cells.

### Example 4

### The Effect of Citrus Polyphenols on the Cytotoxicity of Fibroblast Cells

The LDH test was used to evaluate the cytotoxicity of citrus polyphenols. First, Hs68 cells were cultured according to the method of embodiment 1. After Hs68 cells were cultured with citrus polyphenols for 4 hours, 3 days, and 6 days, respectively, 100 μl/well of supernatant was collected and placed in a new 96 well plate. Subsequently, 100 μl of lactic dehydrogenase (LDH) was added to each well with incubation at 37° C. for 30 minutes and the addition of 50 μl of 1N HCl. ELISA plate reader (M2′, Molecular Devices) was used to detect optical density at 490 nm and 630 nm.

The LDH test results were shown in FIG. 2B. The level of releasing LDH for Hs68 cells which were cultured with 0.01 wt % and 0.1 wt % of citrus polyphenols was less than the Hs68 cells without citrus polyphenols. The differences were more noticeable after 6 days of culturing with citrus polyphenols. These results are consistent with the results of embodiments 2, i.e. high concentration (1 wt %) of citrus polyphenols can inhibit the proliferation of Hs68 cells. On the other hand, citrus polyphenols at 0.01 wt % and 0.1 wt % can maintain the survival of cells without inhibiting cell proliferation.

In combining the results of FIGS. 2A and 2B, they demonstrated that the specific concentrations of citrus polyphenols were able to induce the growth of Hs68 cells without causing cytotoxicity.

### Example 5

### Evaluation the Migration Ability of Fibroblast

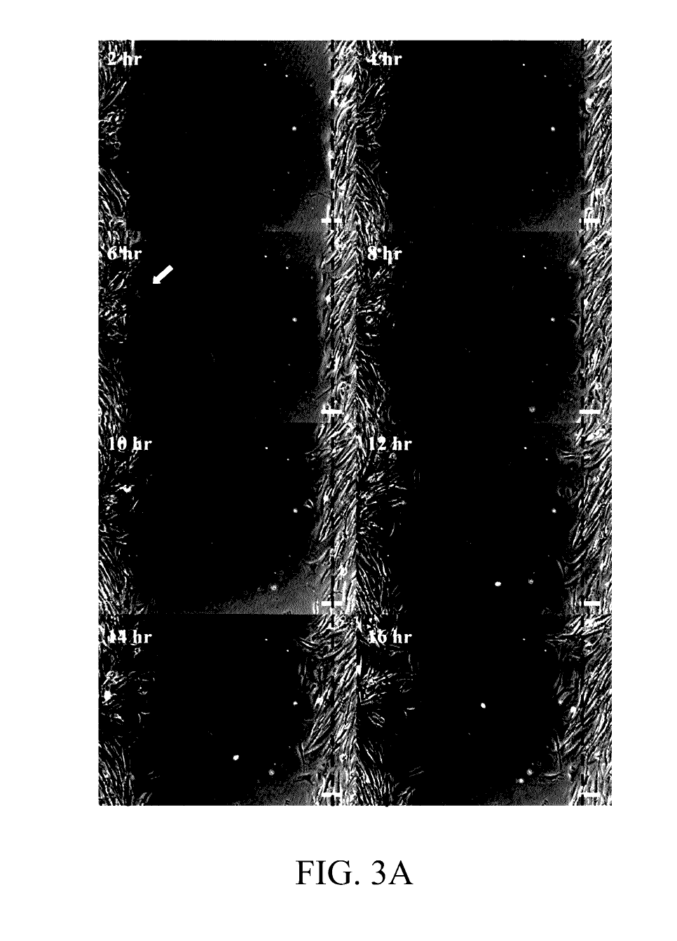

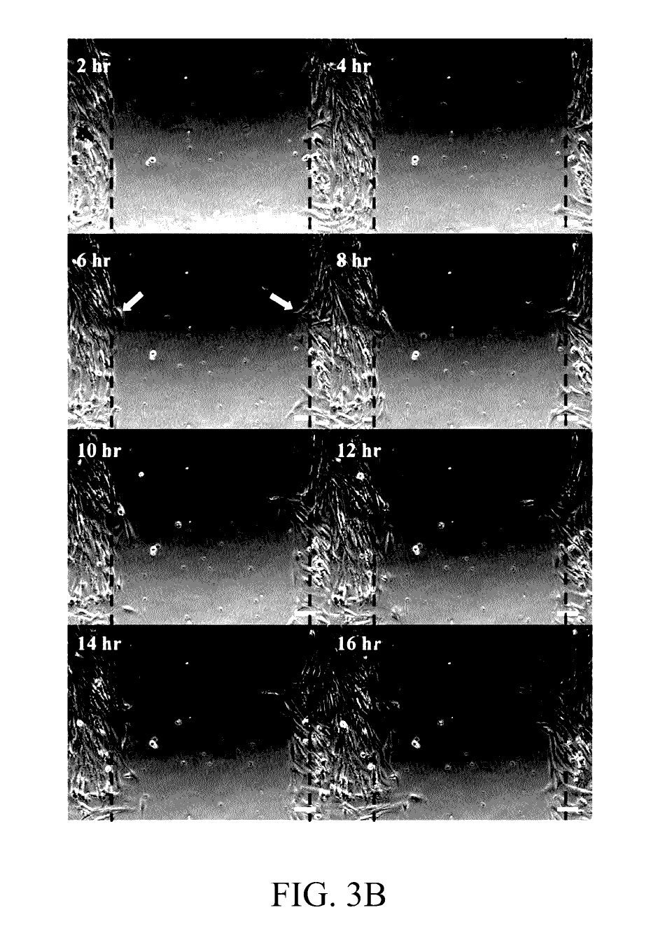

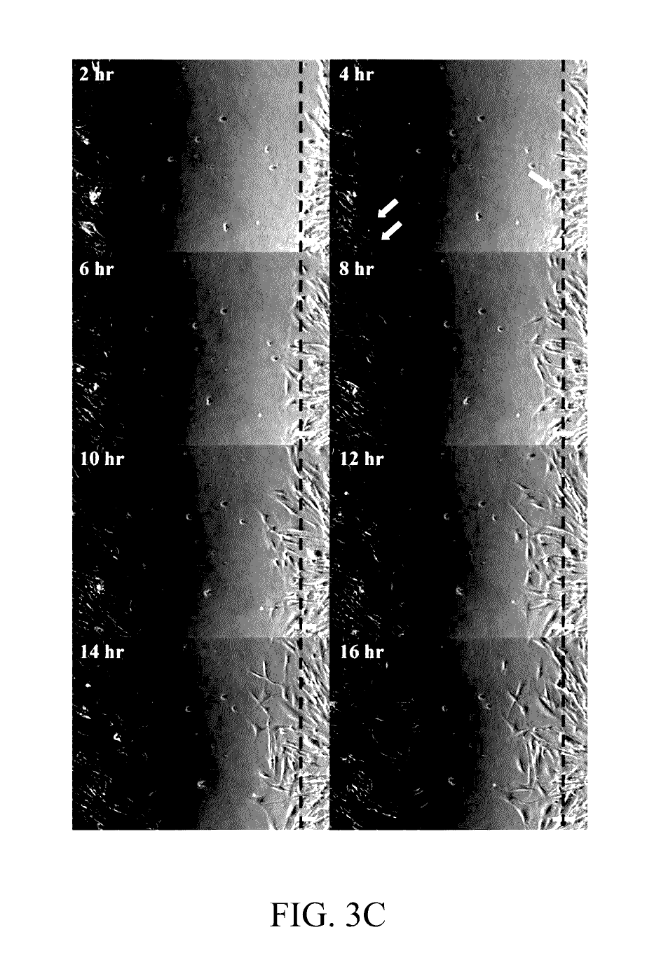

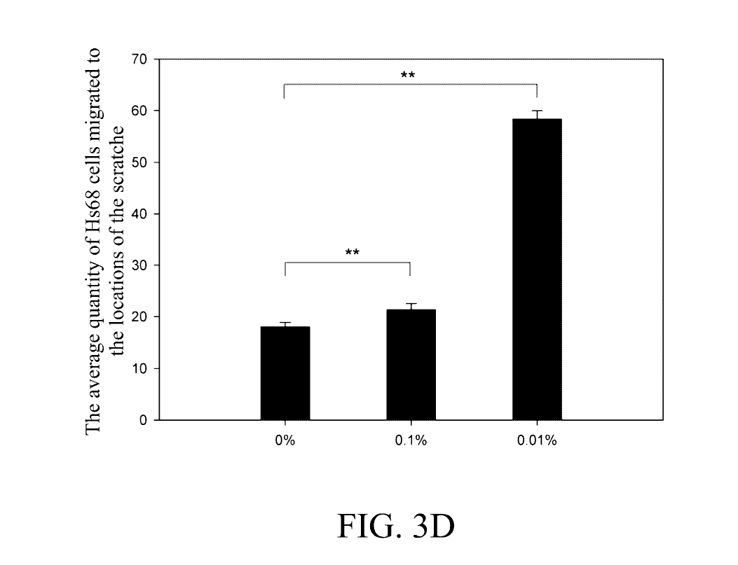

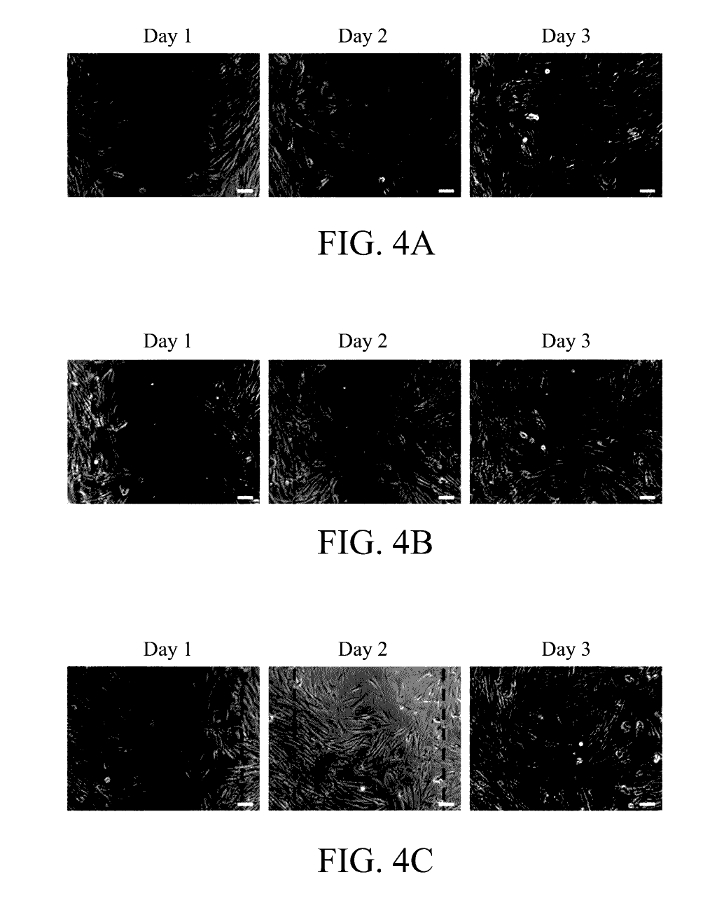

Several studies indicate that fibroblast cells can proliferate and migrate to the locations of the wounds within short period of time. The migration ability of fibroblast is evaluated using scratch assay. For performing scratch assay, Hs68 cells were cultured in DMEM medium containing 10% FBS till confluence. To generate rectangular scratches, used a pipette tip to gently scratch monolayer cells and followed by removing medium and disassociated cells. Used PBS to wash the holes which have been scratched twice and fresh medium with or without citrus polyphenols was added. The cells were cultured 16 hours (short period of time) or 3 days (long period of time). Subsequently, the migration ability of cells was detected using handstand phase contrast time-lapse imaging microscope (Liesa DM1600, Germany). The results were shown in FIG. 3A-4C.

FIG. 3A-3C, respectively, showed the dynamic processes of Hs68 cells during the scratch assay within 16 hours in medium without citrus polyphenols, containing 0.1% citrus polyphenols, or containing 0.01% citrus polyphenols. FIG. 3D showed the average quantity of Hs68 cells which migrated to the location of the wound after 16 hours in media containing different concentrations of citrus polyphenols. FIG. 4A-4C, respectively, showed the images of Hs68 cells under optical microscope during the scratch assay within 3 days in medium without citrus polyphenols, containing 0.1% citrus polyphenols, or containing 0.01% citrus polyphenols. The scratch location was marked within the dotted lines in the figures. The cell numbers which were counted within certain period of time within the dotted lines during the experiment are the numbers of fibroblast cells which proliferated or migrated.

Referring to FIGS. 3A and 3B, the first Hs68 cell (indicated as white arrow) which migrated to the location of the scratches was observed after culturing for 6 hours in the medium without citrus polyphenols or containing 0.1 wt % citrus polyphenols. Referring to FIG. 3C, the first Hs68 cell (indicated as white arrow, 3 locations) which migrated to the location of the scratches was observed after culturing for 4 hours in the medium containing 0.01 wt % citrus polyphenols.

Within the 6-16 hours of the continuous migration of Hs68 cells, there are merely a few cells which migrated to the locations of the scratches (the end point of the observation in the experiment) at the sixteenth hour as shown in FIG. 3A. In addition, in comparing to the medium without citrus polyphenols, relatively more Hs68 cells are mixed-types (polygonal and elongated) in medium containing 0.1 wt % or 0.01 wt % of citrus polyphenols as shown in FIGS. 3B and 3C.

Referring to FIG. 3D, the quantity of Hs68 cells in the locations of the scratches in the medium containing 0 wt %, 0.1% wt %, or 0.01 wt % of citrus polyphenols is 18±1, 21±2, or 58±2, respectively. Therefore, the quantity of Hs68 cells is higher in the locations of the scratches after culturing with citrus polyphenols. In addition, the cell quantity in medium containing 0.01 wt % of citrus polyphenols is three times of the cell quantity in medium containing 0 wt % of citrus polyphenols.

FIG. 4A-4C showed the results of scratch assays after culturing with polyphenols for a longer period of time (3 days). It is found that the growth of Hs68 cells reached full coverage after culturing with 0.01 wt % citrus polyphenols for 2 days. It indicated that fibroblasts migrated gradually to the locations of the scratches, and the migration speed is higher than the controls (not containing citrus polyphenols). Therefore, the treatment of 0.01% citrus polyphenols provided higher efficacy for wound repairing.

Based on the results of this embodiment, Hs68 cell possessed better migration ability to improve wound healing after the treatment of citrus polyphenols, especially the treatment of 0.01 wt % of citrus polyphenols.

### Example 6

### Statistic Analysis

All of the tests have 6 repeated measurements. The data were analyzed using the one-way analysis of variance (ANOVA) and post hoc analysis (Duncan's test) to detect any significant variable among the average values of each treatment.

### Example 7

### Preparation of Local Water-Gel

The composition prepared in one of the embodiments in the present invention is local water-gel for the application of skin wounds or ulcer healing. According to the embodiments of the present invention, preferably the applied dosage of the local water-gel can be once daily, twice daily, three times daily, or depending on needs. In addition, when applied the local water-gel to the wounds or ulcers, the local water-gel contacted the wounds or ulcers and stayed for a period of time.

According to the embodiments of the present invention, the steps of preparation method of the local water-gel are as follows, and the ratios of the components are listed in table 1:

(1) The citrus polyphenols were diluted with water to a desired concentration to obtain solution A;

(2) Polyvinylpyrrolidone or polymer thereof was dissolved in water to obtain solution B;

(3) Solution A was added to solution B with adequate mixing; and

(4) Let the mixed solution of step (3) sit in sterile environment at room temperature for a period of time to obtain the local water-gel containing citrus polyphenols.

TABLE 1
The composition of the local water-gel of the embodiment

| Composition | Content | |
|---|---|---|
| Water | Add to reach 100 wt % | |
| Citrus polyphenols | 0.01-0.1 | wt % |
| Polyvinylpyrrolidone | 2-5 | wt % |
| Propanediol | 1.5-2.5 | wt % |

### Example 8

### Toothpaste Preparation

One composition prepared in one of the embodiments of the present invention is toothpaste to relieve the symptom of wounds or ulcers in the mouth. According to the embodiments of the present invention, preferably the dosage of the toothpaste can be once daily, twice daily, three times daily, or depending on needs.

According to the embodiment in the present invention, the steps of the preparation method are as follows, and the ratios of the components are listed in table 2:

(1). The citrus polyphenols were diluted with water to a desired concentration to obtain solution A;

(2). The wetting agent and other additives were dissolved in water to obtain mixture B;

(3). Solution A was added to solution B with adequate mixing;
(4). The grinding agent was added to the mixture solution of step (3) with stirring to obtain toothpaste containing citrus polyphenols.

TABLE 2
The composition of the toothpaste of the embodiment

| Composition | Content | |
|---|---|---|
| Water | Add to reach 100 wt % | |
| Citrus polyphenols | 0.01-0.1 | wt % |
| Wetting agent | 35 | wt % |
| Grinding agent | 20 | wt % |
| Other additives | 5 | wt % |

### Example 9

### Preparation of Mouthwash

One composition prepared in one of the embodiments of the present invention is mouthwash to relieve the symptom of wounds or ulcers in the mouth. According to the embodiments of the present invention, preferably the dosage of the mouthwash can be once daily, twice daily, three times daily, or depending on needs. Rinse mouth with appropriate amount of mouthwash agent with retention for a period of time prior to spitting it out each time. The retention time can be 10-60 seconds, 20-60 seconds, 30-60 seconds, preferably 30-60 seconds.

According to a preferable embodiment of the present invention, the ratios of the components of the mouthwash were listed in table 3. Mix each component with stiffing to dissolve completely to obtain mouthwash containing citrus polyphenols.

TABLE 3
Components of the mouthwash of the embodiment

| Composition | Content | |
|---|---|---|
| Water | Add to reach 100 wt % | |
| Citrus polyphenols | 0.01-0.1 | wt % |
| Chlorhexidine | 0.01-0.1 | wt % |
| Spices | 0.001-0.01 | wt % |

The present invention demonstrated that the addition of citrus polyphenols in the composition for improving wound healing can effectively induce the proliferation and migration of fibroblasts and improve wound healing by reducing the time required for recovery.

In addition, since the citrus polyphenols of the present invention are used as additives to the conventional pharmaceutical compositions (such as oral ointment, mouth wash, oral additive strip and so on), there are no needs to change the components of the original pharmaceutical compositions. The addition of citrus polyphenols does not affect the effects of the original pharmaceutical composition, and can generate a composition which has multiple-effects in effectively improving wound healing.

The aforementioned embodiments are merely exemplary to illustrate the theory and effects of the present invention, not to limit the scope of the present invention. Any person skilled in the art can modify and change the aforementioned embodiments without violating the spirits and scope of the present invention. Therefore, the scope of the present invention is described as bellow claims.

### SYMBOL DESCRIPTION

None

What is claimed is:

1. A use of a citrus polyphenol for the preparation of a composition to enhance wound healing, wherein the composition comprises an effective amount of the citrus polyphenol and pharmaceutically acceptable carriers.

2. The use of claim 1, wherein the citrus polyphenol includes delphinidin, pelargonidin, peonidin, malvidin, cyaniding, narirutin, naringin, hesperidin, and neohesperidin.

3. The use of claim 1, wherein the wound includes skin injuries, burns, skin ulcer, or oral ulcer.

4. The use of claim 1, wherein the composition is used to improve the proliferation and/or migration of fibroblasts for the enhancement of wound healing.

5. The use of claim 1, wherein the effective amount of the citrus polyphenol is greater than 0 wt % to less than 1 wt %.

6. The use of claim 5, wherein the effective amount of the citrus polyphenol is greater than 0 wt % to less than or equal to 0.1 wt %.

7. The use of claim 6, wherein the effective amount of the citrus polyphenol is 0.01 wt %.

8. A use of a citrus polyphenol for the preparation of a composition to enhance the proliferation and/or migration of fibroblasts, wherein the composition comprises an effective amount of the citrus polyphenol and pharmaceutically acceptable carriers.

9. The use of claim 8, wherein the citrus polyphenol includes delphinidin, pelargonidin, peonidin, malvidin, cyaniding, narirutin, naringin, hesperidin, and neohesperidin.

10. The use of claim 8, wherein the effective amount of the polyphenol is greater than 0 wt % to less than 1 wt %.

11. The use of claim 10, wherein the effective amount of the citrus polyphenol is greater than 0 wt % to less than or equal to 0.1 wt %.

12. The use of claim 11, wherein the effective amount of the citrus polyphenol is 0.01 wt %.

13. A composition for enhancing wound healing, comprising an effective amount of a citrus polyphenol, and pharmaceutically acceptable carriers.

14. The composition of claim 13, wherein the citrus polyphenol includes delphinidin, pelargonidin, peonidin, malvidin, cyaniding, narirutin, naringin, hesperidin, and neohesperidin.

15. The composition of claim 13, wherein the content of the citrus polyphenol is greater than 0 wt % to less than 1 wt % based on the total weight of the composition.

16. The composition of claim 15, wherein the content of the citrus polyphenol is greater than 0 wt % to less than or equal to 0.1 wt % based on the total weight of the composition.

17. The composition of claim 16, wherein the content of the citrus polyphenol is 0.01 wt % based on the total weight of the composition.

18. The composition of claim 13 is a composition for a use in mouth.

19. The composition of claim 18, which is in a dosage form selected from the group consisting of mouthwash, tooth powder, tooth paste, dental gel, periodontal gel, chewable tablet, thin film, oral strip, oral gel, oral tablet, and foaming tablet.

20. The composition of claim 13, wherein the pharmaceutically acceptable carrier is at least one selected from the group consisting of foaming agent, disintegration agent, excipient, viscosity regulator, diluent, surfactant, pH modifier, grinding agent, wetting agent, mouthfeel agent, sweetener, spice, coloring agent, preservative, stabilizer, and anti-bacteria agent.
